(19) United States
(12) Patent Application Publication
(10) Pub. No.: US 2022/0023657 A1
(43) Pub. Date: Jan. 27, 2022

(54) DISPLAYS WITH THERAPEUTIC-LIGHT EMITTERS AND METHODS OF THEIR USE

(71) Applicant: X Display Company Technology Limited, Dublin (IE)

(72) Inventors: Christopher Andrew Bower, Raleigh, NC (US); Matthew Alexander Meitl, Durham, NC (US); Ronald S. Cok, Rochester, NY (US)

(21) Appl. No.: 17/382,275

(22) Filed: Jul. 21, 2021

Related U.S. Application Data
(60) Provisional application No. 63/055,319, filed on Jul. 22, 2020.

(51) Int. Cl. A61N 5/06; G02F 1/13357; G02F 1/1335

(52) U.S. Cl. CPC A61N 5/06; G02F 1/133603; G02F 1/133613; A61N 2005/0626; A61N 2005/0659; A61N 2005/0663; A61N 2005/0652

(57) ABSTRACT

A therapeutic information display comprises an information signal, an array of pixels responsive to the information signal, each pixel in the array of pixels comprising one or more pixel light emitters responsive to the information signal to emit light, and a therapeutic-light emitter. The therapeutic-light emitter emits light having a wavelength in the range of 650 to 1000 nm. A therapeutic front light comprises a cover substrate disposed in relation to an information display and a therapeutic-light emitter disposed on or in the cover substrate, wherein the therapeutic-light emitter emits invisible light having a wavelength no greater than 1000 nm. A therapeutic window comprises a transparent sheet of material and a therapeutic-light emitter comprising one or more quantum dots or phosphors embedded in the transparent sheet of material. The therapeutic-light emitter emits invisible light having a wavelength no greater than 1000 nm.

### PRIORITY APPLICATION

The present application claims the benefit of U.S. Patent Application No. 63/055,319, filed on Jul. 22, 2020, the disclosure of which is hereby incorporated by reference in its entirety.

### TECHNICAL FIELD

The present disclosure relates to displays (e.g., flat-panel micro-LED displays) and windows operable to provide therapeutic light emission.

### BACKGROUND

Flat-panel information displays are widely used throughout the world. Recent research reveals that people spend an average of more than 6 hours each day looking at information displays, and often much more. Such large amounts of time can lead to decreased mental and eye health.

Emissive information displays employ controllable light emitters to display information. For example, liquid crystal displays control light output from a backlight and organic light-emitting diodes use thin films of organic materials that emit light when electrical current passes through the organic films. Light-emitting diode (LED) displays, such as inorganic light-emitting diode displays have many advantages as a display technology, including brightness, efficiency, saturated color emission, and environmental robustness. Most commercially available LED displays comprise relatively large LEDs patterned at a low resolution, for example 25 ppi or having a pitch of approximately one millimeter. More recently, displays with micro-LEDs and much greater resolution have been disclosed. For example, U.S. Pat. No. 8,722,458 entitled “Optical Systems Fabricated by Printing-Based Assembly” teaches transferring light-emitting, light-sensing, or light-collecting semiconductor elements from a wafer substrate to a destination substrate, U.S. Pat. No. 9,991,163 entitled “Small Aperture Ratio Display with Electrical Component” discusses micro-LED displays in conjunction with an electrical component.

Despite the wide range of flat-panel displays available and the increasing quality of images shown on the displays, the amount of time viewers spend looking at screens of all kinds continues to increase and can cause health issues. There is a need therefore for information displays that mitigate the harm that lengthy screen viewing can impose on viewers.

### SUMMARY

The present disclosure provides, inter alia, a therapeutic information display comprises an information signal, an array of pixels responsive to the information signal, each pixel in the array of pixels comprising one or more pixel light emitters responsive to the information signal to emit light, and a therapeutic-light emitter, wherein the therapeutic-light emitter emits light having a wavelength in the range of 650 to 1000 nm. The therapeutic-light emitter can be responsive to a control signal separate from the information signal or the therapeutic-light emitter can be responsive to the information signal. According to some embodiments, the wavelength of the therapeutic-light emitter is substantially 670 nm or substantially 830 nm.

The therapeutic information display can comprise a plurality of therapeutic-light emitters. The number of therapeutic-light emitters can be equal to the number of pixels or can be less than the number of pixels.

The therapeutic information display can comprise a display substrate, the pixel light emitters can be disposed on the display substrate, and the therapeutic-light emitter can be disposed on the display substrate. In some embodiments, the therapeutic information display comprises a display substrate and a cover substrate, the pixel light emitters are disposed on the display substrate, and the therapeutic-light emitter is disposed on the cover substrate.

According to some embodiments, the information display is a liquid-crystal display comprising a backlight and the therapeutic-light emitter is disposed in the backlight. According to some embodiments the information display is an inorganic light-emitting diode display or an organic light-emitting diode display. According to some embodiments, the therapeutic-light emitter is an inorganic light emitter.

According to some embodiments, the therapeutic-light emitter comprises a phosphor or quantum dot that absorbs light having a wavelength shorter than the wavelength of light emitted by the therapeutic-light emitter. According to some embodiments, the therapeutic information display comprises a color filter and the phosphor or quantum dot is disposed in or on the color filter. According to some embodiments, the therapeutic information display comprises a display cover substrate and the phosphor or quantum dot is disposed in or on the cover substrate. According to some embodiments, the therapeutic-light emitter comprises a light-emitting diode and the phosphor or quantum dot is disposed over or on the light-emitting diode.

According to some embodiments, the therapeutic light is subliminal or invisible, wherein the therapeutic light is emitted with a subliminal intensity, or the therapeutic light is emitted with a duty cycle less than one, no greater than one half, no greater than one quarter, no greater than one tenth, no greater than one twentieth, no greater than 4%, no greater than 3%, no greater than 2%, no greater than 1.66%, no greater than 1.43%, no greater than 1.33% or no greater than 1%.

The therapeutic-light emitter can be electrically connected in series or in parallel with any one or all of the one or more pixel light emitters.

According to some embodiments, the therapeutic-light emitter emits red light and the display comprises a controller that controls the therapeutic-light emitter to emit red light having an intensity equal to the least intensity of red light emitted by any pixel light emitter in a display area illuminated by the therapeutic-light emitter as specified by the information signal. According to some embodiments, the therapeutic-light emitter comprises a controller that controls the therapeutic-light emitter to emit light only when one or more pixel light emitters are controlled to emit red light.

According to embodiments of the present disclosure, a therapeutic information display comprises an array of pixels that display information, each pixel in the array of pixels comprising one or more pixel light emitters and one of the pixel light emitters is a therapeutic-light emitter. The therapeutic-light emitter emits light having a wavelength between 650 and 1000 nm. Each pixel can comprise a red-light emitter and the red-light emitter can be a therapeutic-light emitter. Each pixel can comprise a therapeutic-light emitter and a red-light emitter in addition to the therapeutic-light emitter.

According to embodiments of the present disclosure, a therapeutic front light comprises a cover substrate disposed in relation to an information display and a therapeutic-light emitter is disposed on or in the cover substrate, wherein the therapeutic-light emitter emits invisible light having a wavelength no greater than 1000 nm.

According to embodiments of the present disclosure, a therapeutic window comprises a transparent sheet of material and a therapeutic-light emitter comprising one or more quantum dots or phosphors embedded in the transparent sheet of material, wherein the therapeutic-light emitter emits invisible light having a wavelength no greater than 1000 nm.

In some embodiments, a therapeutic information display comprises an array of pixels, each pixel in the array of pixels comprising one or more pixel light emitters responsive to an information signal to emit light; and a therapeutic-light emitter (e.g., comprising an organic or inorganic light emitting diode), wherein the therapeutic-light emitter emits light having a wavelength in a range of 650 nm to 1000 nm [e.g., wherein the display is operable to emit a therapeutically effective amount of light (e.g., as administered in one or more unit doses, e.g., administered periodically, optionally, within a certain period and/or separated by at least a certain length of time) from the therapeutic-light emitter having a wavelength in a range of 650 nm to 1000 nm)].

In some embodiments, a method of operating a display to provide a therapy to a subject includes providing the display comprising one or more therapeutic-light emitters; and displaying information on the display, wherein displaying the information causes a therapeutically effective amount of light to be emitted from the one or more therapeutic-light emitters. The one or more therapeutic-light emitters can be included in a backlight or a front light of the display, or in one or more pixels in the display. The information displayed can be one or more videos (e.g., comprising a television show or film) or an image (e.g., text).

In some embodiments, a method of operating a display to provide a therapy to a subject includes providing the display comprising one or more therapeutic-light emitters; and displaying information on the display and simultaneously emitting a therapeutically effective amount of light from the one or more therapeutic-light emitters. The one or more therapeutic-light emitters can be included in a backlight or a front light of the display, or in one or more pixels in the display. The information displayed can be one or more videos (e.g., comprising a television show or film) or an image (e.g., text). In some embodiments, a method for treating and/or preventing a condition in a subject includes administering light therapy to a subject by directing light predominately (e.g., at least 50%, at least 80%, or at least 90%) or solely having one or more wavelengths in a range from 650 nm to 1000 nm into an eye (e.g., cornea or retina) and/or onto a skin of a subject by a display (e.g., a backlight or front light of the display). The light can consist essentially of light having a wavelength of substantially 670 nm and/or substantially 830 nm. The therapeutic light can be administered in combination with a separate pharmaceutical composition, for example given as an ocular or topical administration. The condition can be inflammation, cell degeneration, visual decline (e.g., age-related vision loss), glaucoma, optic nerve injury, ocular implant recovery, increased intraocular pressure, retinitis pigmentosa, or seasonal affective disorder. Administration can occur while the subject receives information (e.g., watches a television show or film or reads text) from the display.

In some embodiments, a method for stimulating generation of adenosine triphosphate (ATP) in a subject includes administering light (e.g., light therapy) to a subject by directing light predominately (e.g., at least 50%, at least 80%, or at least 90%) or solely having one or more wavelengths in a range from 650 nm to 1000 nm into an eye (e.g., cornea or retina) of a subject by a display (e.g., a backlight or front light of the display) such that a therapeutically effective amount of light is delivered to the subject to stimulate the generation of ATP. The light can consist essentially of light having a wavelength of substantially 670 nm and/or substantially 830 nm. The therapeutic light can be administered in combination with a separate pharmaceutical composition, for example given as an ocular or topical administration. In some embodiments, administration occurs while the subject receives information (e.g., watches a television show or film or reads text) from the display.

In some embodiments of these methods, emitting the therapeutically effective amount of light from only one or more portions of the display that are displaying information that comprises a red component. In some embodiments of these methods, emitting the therapeutically effective amount of light from one or more of the one or more therapeutic light emitters that correspond to one or more red light emitters that are emitting light to display the information. In some embodiments of these methods, emitting the therapeutically effective amount of light at a lower intensity than light emitted to display the information (e.g., than an intensity of red light emitted to display the information). In some embodiments of these methods, emitting therapeutically effective amount of light at a frequency lower than a refresh rate of the display (e.g., less than 120 Hz, 60 Hz, or 30 Hz).

The present disclosure provides, inter alia, flat-panel information displays, a therapeutic front light, and a therapeutic window that emit light having therapeutic value to humans viewing the displays.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 is a schematic plan view and detail of a therapeutic information display according to some illustrative embodiments of the present disclosure;

FIGS. 2 and 3 are schematic circuits of a therapeutic information display according to some illustrative embodiments of the present disclosure;

FIG. 4 is a schematic plan view and detail of a therapeutic information display according to some illustrative embodiments of the present disclosure;

FIGS. 5A and 5B are schematic cross sections of a therapeutic-light emitter according to some illustrative embodiments of the present disclosure;

FIG. 6 is a cross section of a therapeutic information display comprising a display substrate and a cover substrate according to some illustrative embodiments of the present disclosure;

FIG. 7 is a cross section of a therapeutic information display comprising a backlight and a liquid crystal display according to some illustrative embodiments of the present disclosure;

FIG. 8 is a schematic illustration of pixel light emitters electrically connected with a therapeutic-light emitter according to some illustrative embodiments of the present disclosure;

FIG. 9 is a schematic plan view of a therapeutic window according to some illustrative embodiments of the present disclosure; and

FIG. 10 is a schematic plan view of a therapeutic window comprising an array of therapeutic-light emitters each comprising a light emitting diode (e.g., an organic or inorganic light emitting diode) and electrically connected using a plurality of wires or traces, according to some embodiments of the present disclosure.

Features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements. The figures are not drawn to scale since the variation in size of various elements in the Figures is too great to permit depiction to scale.

### Definitions

Administration: As used herein, the term “administration” typically refers to the administration of a therapy (e.g., a light therapy) and/or a composition (e.g., photon) to a subject or system, for example to expose one or more parts of the body of a subject to the therapy (e.g., to expose the eyes (or part thereof), skin, or other body part of the subject to a particular kind of light) and/or to achieve delivery of an agent that is, is included in, or is otherwise delivered by, the composition. Non-limiting examples of administration of a therapy and/or composition to a subject include parenteral administration (for example, by subcutaneous, intramuscular, intravenous or epidural exposure); topical application (for example, as incident energy (e.g., photons)); and ocular administration.

Agent: As used herein, the term “agent” refers to an entity, for example a photon or a phenomenon such as light (e.g., a particular kind of light).

Amelioration: As used herein, the term “amelioration” refers to the prevention, reduction, palliation, or improvement of a state of a subject. Amelioration includes, but does not require, complete recovery or complete prevention of a disease, disorder or condition.

Improved, increased, or reduced: As used herein, these terms, or grammatically comparable comparative terms, indicate values that are relative to a comparable reference measurement. For example, in some embodiments, e.g., as set forth herein, an assessed value achieved with an agent of interest may be “improved” relative to that obtained with a comparable reference agent or with no agent. Alternatively or additionally, in some embodiments, e.g., as set forth herein, an assessed value in a subject or system of interest may be “improved” relative to that obtained in the same subject or system under different conditions or at a different point in time (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, e.g., as set forth herein, comparative terms refer to statistically relevant differences (e.g., differences of a prevalence and/or magnitude sufficient to achieve statistical relevance). Those of skill in the art will be aware, or will readily be able to determine, in a given context, a degree and/or prevalence of difference that is required or sufficient to achieve such statistical significance.

Pharmaceutical composition: As used herein, the term “pharmaceutical composition” refers to a composition in which an active agent is provided together with one or more pharmaceutically acceptable carriers. In some embodiments, e.g., as set forth herein, the active agent is present in a unit dose amount appropriate for administration to a subject, e.g., in a therapeutic regimen that shows a statistically significant probability of achieving a predetermined therapeutic effect when administered to a relevant population. In some embodiments, e.g., as set forth herein, a pharmaceutical composition can be formulated for administration in a particular form (e.g., in a solid form or a liquid form), and/or can be specifically adapted for, for example: oral administration (for example, as a drenche (aqueous or non-aqueous solutions or suspensions), tablet, capsule, bolus, powder, granule, paste, etc., which can be formulated specifically for example for buccal, sublingual, or systemic absorption); parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Prevent or prevention: The terms “prevent” and “prevention,” as used herein in connection with the occurrence of a disease, disorder, or condition, refers to reducing the risk of developing the disease, disorder, or condition; delaying onset of the disease, disorder, or condition; delaying onset of one or more characteristics or symptoms of the disease, disorder, or condition; and/or to reducing the frequency and/or severity of one or more characteristics or symptoms of the disease, disorder, or condition. Prevention can refer to prevention in a particular subject or to a statistical impact on a population of subjects. Prevention can be considered complete when onset of a disease, disorder, or condition has been delayed for a predefined period of time.

Prognosis: As used herein, the term “prognosis” refers to determining the qualitative or quantitative probability of at least one possible future outcome or event. As used herein, a prognosis can be a determination of the likely course of a disease, disorder, or condition such as cancer in a subject, a determination regarding the life expectancy of a subject, or a determination regarding response to therapy, e.g., to a particular therapy.

Sample: As used herein, the term “sample” typically refers to an aliquot of material obtained or derived from a source of interest. In some embodiments, e.g., as set forth herein, a source of interest is a biological or environmental source. In some embodiments, e.g., as set forth herein, a sample is a “primary sample” obtained directly from a source of interest. In some embodiments, e.g., as set forth herein, as will be clear from context, the term “sample” refers to a preparation that is obtained by processing of a primary sample (e.g., by removing one or more components of and/or by adding one or more agents to a primary sample).

Susceptible to: An individual or subject who is “susceptible to” a disease, disorder, or condition is at risk for developing the disease, disorder, or condition. In some embodiments, e.g., as set forth herein, an individual who is susceptible to a disease, disorder, or condition does not display any symptoms of the disease, disorder, or condition. In some embodiments, e.g., as set forth herein, an individual who is susceptible to a disease, disorder, or condition has not been diagnosed with the disease, disorder, and/or condition. In some embodiments, e.g., as set forth herein, an individual who is susceptible to a disease, disorder, or condition is an individual who has been exposed to conditions associated with, or presents a biomarker status associated with, development of the disease, disorder, or condition. In some embodiments, e.g., as set forth herein, a risk of developing a disease, disorder, and/or condition is a population-based risk (e.g., family members of individuals suffering from the disease, disorder, or condition).

Subject: As used herein, the term “subject” refers to an organism, typically a mammal (e.g., a human). In some embodiments, e.g., as set forth herein, a subject is suffering from a disease, disorder or condition. In some embodiments, e.g., as set forth herein, a subject is susceptible to a disease, disorder, or condition. In some embodiments, e.g., as set forth herein, a subject displays one or more symptoms or characteristics of a disease, disorder or condition. In some embodiments, e.g., as set forth herein, a subject is not suffering from a disease, disorder or condition. In some embodiments, e.g., as set forth herein, a subject does not display any symptom or characteristic of a disease, disorder, or condition. In some embodiments, e.g., as set forth herein, a subject is someone with one or more features characteristic of susceptibility to or risk of a disease, disorder, or condition. In some embodiments, e.g., as set forth herein, a subject is a patient. In some embodiments, e.g., as set forth herein, a subject is an individual to whom diagnosis has been performed and/or to whom therapy has been administered. In some instances, e.g., as set forth herein, a human subject can be interchangeably referred to as an “individual.”

Therapeutic agent, pharmaceutical agent, and active agent: As used herein, the terms “therapeutic agent”, “pharmaceutical agent”, and “active agent” are interchangeable, and each refers to any agent that elicits a desired beneficial (e.g., therapeutic and/or pharmacological) effect when administered to a subject. In some embodiments, e.g., as set forth herein, an agent is considered to be a therapeutic agent if it demonstrates a statistically significant effect across an appropriate population. In some embodiments, e.g., as set forth herein, the appropriate population can be a population of model organisms or a human population. In some embodiments, e.g., as set forth herein, an appropriate population can be defined by various criteria, such as a certain age group, gender, genetic background, preexisting clinical conditions, etc. In some embodiments, e.g., as set forth herein, a therapeutic agent is a therapy or substance that can be used for treatment of a disease, disorder, or condition. In some embodiments, e.g., as set forth herein, a therapeutic agent is an agent that has been or is required to be approved by a government agency before it can be marketed for administration to humans. In some embodiments, e.g., as set forth herein, a therapeutic agent is an agent for which a medical prescription is required for administration to humans. In some embodiments, light (e.g., one or more photons) is a therapeutic agent.

Therapeutically effective amount: As used herein, the term “therapeutically effective amount” refers to an amount (e.g., of therapy and/or a composition) that produces a desired effect for which it is administered. In some embodiments, e.g., as set forth herein, the term refers to an amount that is sufficient, when administered to a population suffering from or susceptible to a disease, disorder, or condition, in accordance with a therapeutic treatment and/or dosing regimen, to treat the disease, disorder, or condition. Those of ordinary skill in the art will appreciate that the term therapeutically effective amount does not in fact require successful treatment be achieved in a particular individual. Rather, a therapeutically effective amount can be an amount that provides a particular desired therapeutic and/or pharmacological response in a significant number of subjects when administered to individuals in need of such treatment. In some embodiments, e.g., as set forth herein, reference to a therapeutically effective amount can be a reference to an amount of a composition as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent can be formulated and/or administered in a single dose or session. In some embodiments, e.g., as set forth herein, a therapeutically effective agent can be formulated and/or administered in a plurality of doses or sessions, for example, as part of a multi-dose dosing regimen and/or a multi-session treatment therapy (e.g., light therapy).

Treatment: As used herein, the term “treatment” (also “treat” or “treating”) refers to administration of a therapy (e.g., light therapy) that partially or completely alleviates, ameliorates, relieves, inhibits, delays onset of, halts progression of, slows progression of, reverses progression of, reduces severity of, and/or reduces incidence of one or more symptoms, features, and/or causes of a particular disease, disorder, or condition, or is administered for the purpose of achieving any such result. In some embodiments, e.g., as set forth herein, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, e.g., as set forth herein, treatment can be of a subject who has been diagnosed as suffering from the relevant disease, disorder, and/or condition. In some embodiments, e.g., as set forth herein, treatment can be of a subject known to have one or more susceptibility factors that are statistically correlated with increased risk of development of the relevant disease, disorder, or condition. In various examples, treatment is of a cancer.

Unit dose: As used herein, the term “unit dose” refers to an amount of a composition (e.g., photons) and/or therapy (e.g., light) administered as a single dose and/or in a physically discrete unit. In many embodiments, e.g., as set forth herein, a unit dose contains a predetermined quantity of an active agent. In some embodiments, e.g., as set forth herein, a unit dose contains an entire single dose of the agent. In some embodiments, e.g., as set forth herein, more than one-unit dose is administered to achieve a total single dose. In some embodiments, e.g., as set forth herein, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic agent(s). For example, acceptable carriers, diluents, stabilizers, buffers, preservatives, etc., can be included. It will be appreciated by those skilled in the art, in many embodiments, e.g., as set forth herein, a total appropriate daily dosage of a particular therapeutic agent can comprise a portion, or a plurality, of unit doses, and can be decided, for example, by a medical practitioner within the scope of sound medical judgment. In some embodiments, e.g., as set forth herein, the specific effective dose level for any particular subject or organism can depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts. In some embodiments, a unit dose refers to administration of light of one or more of (i) a certain flux (e.g., intensity) (e.g., above a threshold) for a certain duration. Flux (e.g., intensity) of light may be controlled based on a duty cycle of light emitted by a light emitter during its administration, for example where an emitter is cycled at rate that exceeds that which can be perceived by a subject (e.g., faster than ˜0.4 msec for a human).

### DETAILED DESCRIPTION OF CERTAIN EMBODIMENTS

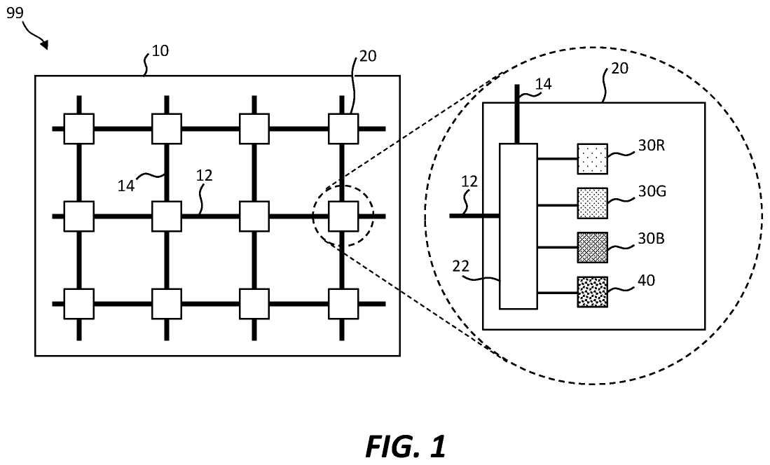

In some embodiments of the present disclosure, inter alia, and referring to the cross section of FIG. 1, an information display 99 comprises an array of pixels 20 responsive to an information signal, for example an information signal carried on row wires 12 and column wires 14 in a matrix-addressed array of pixels 20. The row wires 12 and column wires 14 can be connected to row and column controllers and controlled by a display controller (not shown). Each pixel 20 in the array of pixels 20 comprises one or more pixel light emitters 30 (e.g., red pixel light emitter 30R that emits red light, green pixel light emitter 30G that emits green light, and blue pixel light emitter 30B that emits blue light, collectively pixel light emitters 30) and is responsive to the information signal to emit light. The information display 99 comprises a therapeutic-light emitter 40 that emits light having a wavelength in the range of 650 to 1000 nm (e.g., in the range of 700 to 1000 nm, in the range of 750 to 1000 nm, or in the range of 800 to 1000 nm). Such light, for example light having a frequency of substantially 670 nm (e.g., having a central emission wavelength within 2%, 1.5%, 1%, 0.5%, or 0.25% of 670 nm or wherein at least 80% of light emitted by a light emitter has a wavelength of within 2%, 1.5%, 1%, 0.5%, or 0.25% of 670 nm), substantially 830 nm (e.g., having a central emission wavelength within 2%, 1.5%, 1%, 0.5%, or 0.25% of 830 nm or wherein at least 80% of light emitted by a light emitter has a wavelength of within 2%, 1.5%, 1%, 0.5%, or 0.25% of 830 nm), or having a central frequency of substantially 670 nm or 830 nm and a distribution of frequencies centered about 670 nm or 830 nm, can be therapeutic light that has a beneficial effect on a viewer's eyes. By substantially is meant within the tolerances of a manufacturing process and does not imply that every therapeutic-light emitter 40 emits light that is 670 nm or 830 nm. Rather, therapeutic-light emitter 40 can comprise a distribution of light emitters or converters that are approximately centered on a desired central frequency, for example within 1%, within 2% within 5%, within 10%, within 20%, or within 50%. Pixel light emitters 30 emit light when provided with electrical power, for example under the control of pixel controller 22 to display information, for example an image, on information display 99.

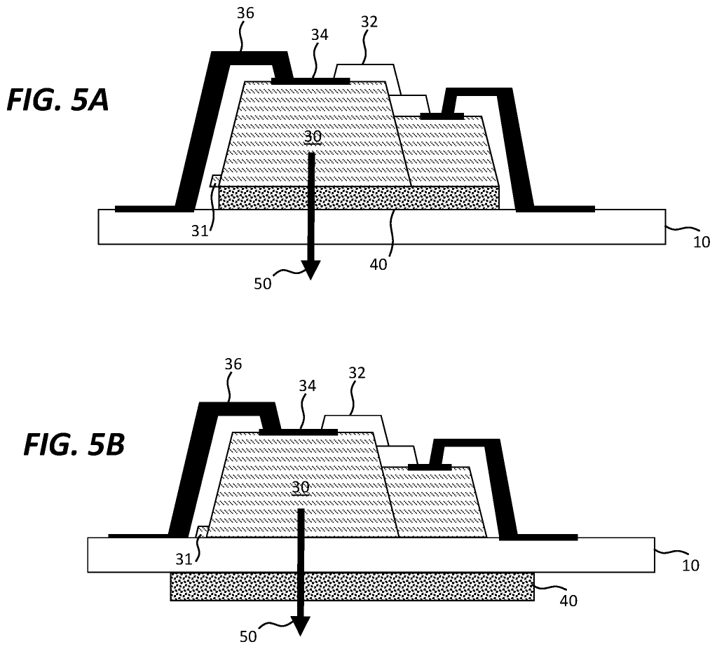

Any one or more of pixels 20 (e.g., comprising pixel controller 22, pixel light emitters 30 and therapeutic-light emitter 40) can be disposed directly on a display substrate 10. In some embodiments, the pixel elements (e.g., pixel controller 22 and pixel light emitters 30) can be provided on a pixel substrate separate and independent from display substrate 10, as can therapeutic-light emitter 40 (not shown). Pixel controller 22 and pixel light emitters 30 (e.g., light-emitting diodes) can be integrated circuits, can be assembled using micro-transfer printing methods, and/or can comprise broken (e.g., fractured) or separated tethers as a consequence of micro-transfer printing. In the case that therapeutic-light emitter 40 is a light-emitting diode, therapeutic-light emitter 40 can comprise a fractured light-emitter tether 31 as a consequence of micro-transfer printing (e.g., as shown in FIGS. 5A and 5B).

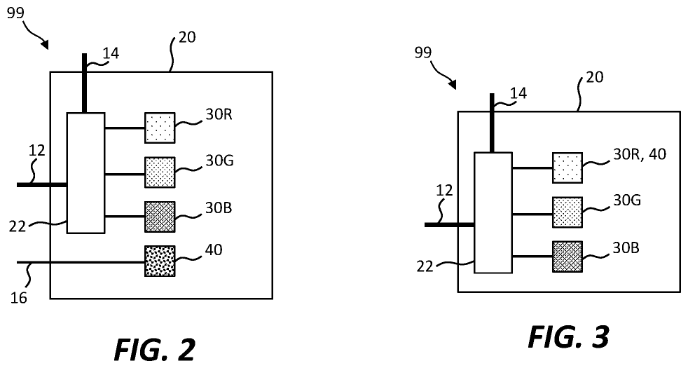

Therapeutic-light emitter 40 can be an active light-emitter, for example a light-emitting diode controlled with electrical power provided by pixel controller 22 (e.g., as shown in FIGS. 1 and 3) or provided through a control signal 16, as shown in FIG. 2. Therapeutic-light emitter 40 can be a light emitter that is separate from any pixel light emitters 30 (as shown in FIGS. 1 and 2) or, as shown in FIG. 3, therapeutic-light emitter 40 can be a pixel light emitter 30 or comprise pixel light emitter 30. Therapeutic-light emitter 40 can be or comprise any one, any combination of, or all of pixel light emitter 30. According to embodiments, therapeutic information display 99 comprises a plurality of therapeutic-light emitters 40. In one embodiment, therapeutic-light emitter 40 is red pixel light emitter 30R.

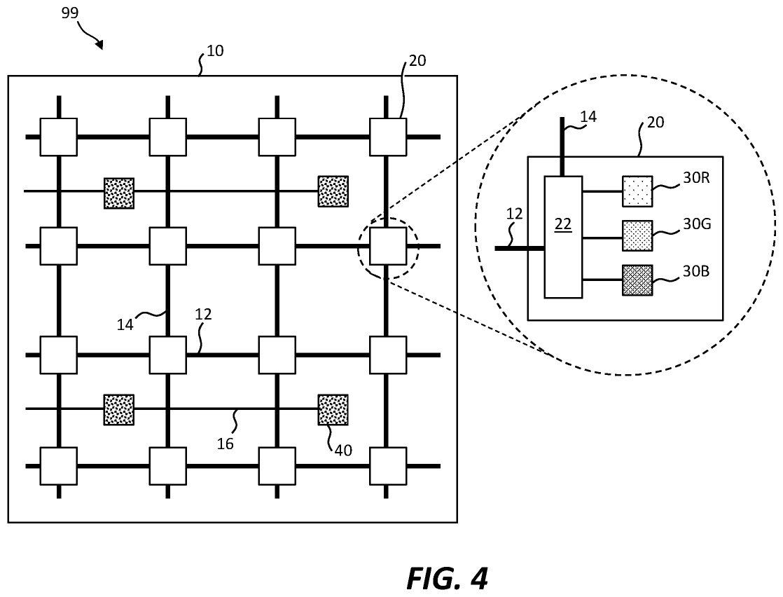

According to some embodiments of the present disclosure, each pixel 20 comprises a therapeutic-light emitter 40, or multiple therapeutic-light emitters 40, so that the number of therapeutic-light emitters 40 is equal to the number of pixels 20 or equal to the number of pixel light emitters 30. As shown in FIG. 4, the number of therapeutic-light emitters 40 is less than the number of pixels 20 and can be controlled by control signal 16 separate from row wires 12 or column wires 14, for example by a display controller (not shown).

According to some embodiments of the present disclosure, therapeutic-light emitter 40 is a passive light emitter that responds to optical stimulation, for example absorbing light of a higher frequency and re-emitting it at a lower frequency, and comprising, for example, phosphors or quantum dots. Such phosphors or quantum dots can be disposed in a liquid carrier, e.g., by coating, and dried to form a layer. As shown in FIGS. 5A and 5B, a light-emitting diode (e.g., a pixel light emitter 30 or a pump light emitter separate from pixels 20) emits light 50 that is absorbed by a layer of phosphors or quantum dots comprising therapeutic-light emitter 40. The phosphors or quantum dots then re-emit the absorbed light at a lower frequency, thereby converting the light from a higher frequency to a lower frequency, for example light having a wavelength in the range of 650 to 1000 nm. According to some embodiments, therapeutic-light emitter 40 is disposed between pixel light emitter 30 and display substrate 10 (or a cover substrate 60, not shown) or is disposed between pixel light emitter 30 and a pixel substrate (not shown). According to some embodiments, a substrate (e.g., display substrate 10, cover substrate 60, or a pixel substrate, not shown) is disposed between therapeutic-light emitter 40 pixel and light emitter 30. Therapeutic-light emitter 40 can also be stimulated by a light-emitting diode that is separate and independent of any pixel light emitter 30, for example as shown in FIG. 4.

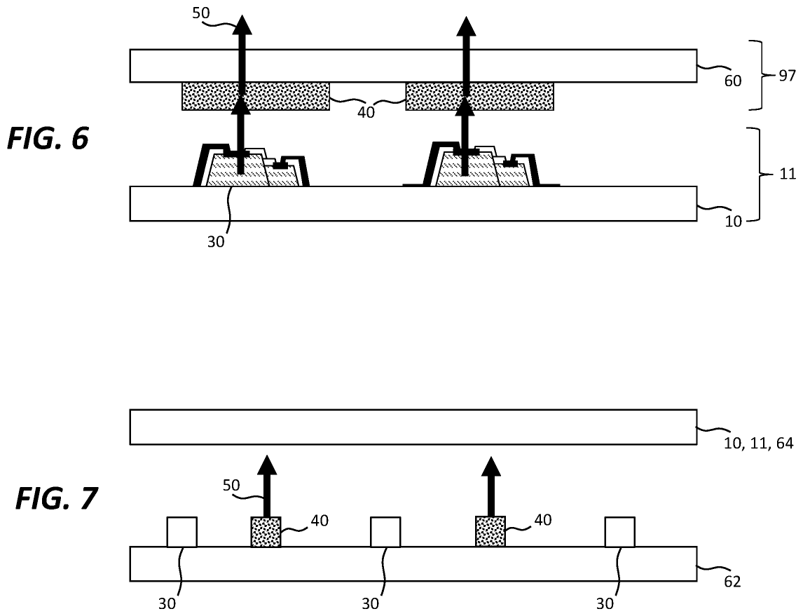

Therapeutic-light emitter 40 can be disposed on a common substrate with pixel light emitters 30. According to some embodiments, therapeutic-light emitter 40 is disposed on a substrate separate and independent of a substrate (e.g., separate and independent of display substrate 10), for example disposed on a cover substrate 60 disposed over display substrate 10, as shown in FIG. 6. Therapeutic-light emitter 40 can be an active light emitter (e.g., a light-emitting diode) or a passive light emitter (e.g., a coating of phosphors or quantum dots) disposed, for example, on a side of cover substrate 60 between cover substrate 60 and display substrate 10. Cover substrate 60 can be a substrate for a front light and the front light can comprise therapeutic-light emitter 40 as well as light emitters for illuminating an information display (for example an LCD or OLED display). Thus, a therapeutic front light 97 can comprise a cover substrate 60 disposed in relation to an information display 11. Therapeutic-light emitter 40 can emit invisible light having a wavelength no greater than 1000 nm. A passive therapeutic-light emitter 40 can be disposed on cover substrate 60 and can be an unpatterned coating. According to some embodiments, cover substrate 60 is adhered to information display 11, for example as an overlay. Cover substrate 60 can be substantially transparent, for example no less than 50% transparent to visible light and to therapeutic light (e.g., no less than 70% transparent, no less than 80% transparent, or no less than 90% transparent to visible light and to therapeutic light).

According to some embodiments, therapeutic-light emitter 40 can be a passive light emitter that is incorporated into a color filter of an information display.

As shown in FIG. 7, therapeutic-light emitter 40 can be disposed in a back light 62 of a liquid crystal display 64 so that light 50 emitted through the liquid crystal display 64 is seen by a viewer. Therapeutic-light emitter 40 can be an active light emitter that directly emits therapeutic light or a passive light emitter that absorbs light from the backlight and converts the absorbed light to therapeutic light.

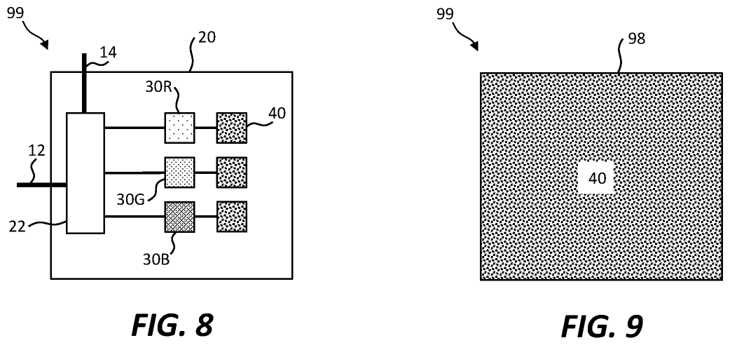

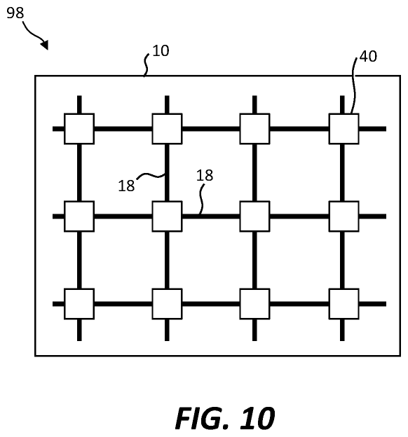

As illustrated in FIG. 8, therapeutic-light emitter 40 can be electrically connected in series or in parallel with any one or combination of pixel light emitters 30. Thus, every time a pixel light emitter 30 is controlled (e.g., by pixel controller 22 in response to an information signal communicated by row wires 12 and column wires 14) to emit light, therapeutic light is also emitted.

According to some embodiments of the present disclosure, therapeutic light emitted by therapeutic-light emitter 40 is invisible light, for example infrared light. Such light is undetected by the human visual system and can be emitted in conjunction with pixel light without having an effect on an image shown on therapeutic information display 99. Invisible therapeutic light can be emitted directly by an LED or by color conversion from a pixel light emitter 30 or from a separately controlled LED. If the invisible therapeutic light is converted by pixel light from any one or more of the pixel light emitters 30, the intensity of the pixel light can be adjusted to accommodate the loss of energy due to the conversion of pixel light to invisible therapeutic light.

According to some embodiments of the present disclosure, therapeutic light emitted by therapeutic-light emitter 40 is visible light, for example red light. The red therapeutic light can be emitted at a level that is not noticeable to a viewer and is below the level of consciousness, for example subliminal light. Subliminal red therapeutic light can be emitted directly by an LED or by color conversion from a pixel light emitter 30 or a separately controlled LED. If the subliminal red therapeutic light is converted by pixel light from any one or more of the pixel light emitters 30, the intensity of the pixel light can be adjusted to accommodate the loss of energy due to the conversion of pixel light to invisible therapeutic light. Subliminal red therapeutic light can be controlled by intensity and by duty cycle, for example emitting a greater intensity of light for shorter periods of time.

A therapeutically effective amount of light can be emitted from only one or more portions of a display that are displaying information that comprises a red component. A therapeutically effective amount of light may be emitted from one or more therapeutic light emitters that correspond to one or more red light emitters that are emitting light to display information. That is, in some embodiments, therapeutic light emitter(s) are disposed in spatial correspondence with red light emitter(s), for example included and electrically connected in pixels in a display. In some embodiments, a therapeutically effective amount of light is emitted at a lower intensity than light emitted to display information (e.g., than an intensity of red light emitted to display the information). Therapeutically effective amount of light can be emitted at a frequency lower than a refresh rate of the display (e.g., less than 120 Hz, 60 Hz, or 30 Hz).

Duty cycle of light provided by a therapeutic-light emitter 40 may be controlled independently of light provided by other emitters in a display 99, for example, in some embodiments, therapeutic-light emitters included in a backlight or front light can be controlled independently from other light emitters in the backlight or front light, for example to have a different duty cycle. Controlling duty cycle of therapeutic-light emitters can be used to control treatment and/or administration of a therapeutically effective amount of light (e.g., red light).

In some embodiments, a light therapy is administered by a display 99. In some embodiments, a therapeutically effective amount of a light therapy can be administered over a period of time of viewing a display 99 of, for example, at least 1 minute (e.g., at least 5 minutes) and, optionally, no more than two hours (e.g., 90 minutes, 60 minutes, or 30 minutes). For example, in some embodiments, a therapeutically effective amount of light can be administered to a subject by a display 99 within the period of time it takes to read a page, article, or chapter of a book or within the length of a video advertisement, television program, film, or other video. In some embodiments, a display 99 is included in a computer (e.g., as in a laptop or tablet) or connected to a computer (e.g., as a desktop). In some embodiments, a therapeutically effective amount of light is provided to a subject by a backlight or front light of a display 99 while the display is turned on (e.g., and providing information to the subject). In some embodiments, a therapeutically effective amount of light is provided by light used to convey information to a subject (e.g., if one or more (e.g., no more than 50% or all) pixels of a display 99 include a pixel light-emitter 30 that is also a therapeutic-light emitter 40). In some embodiments, administration of light to an eye (e.g., a cornea or retina) can stimulate generation of adenosine triphosphate (ATP), which may treat, for example, vision loss.

In some embodiments, a method for treating and/or preventing a condition in a subject comprises administering light therapy to a subject by directing light predominately (e.g., at least 50%, at least 80%, or at least 90%) or solely having one or more wavelengths in a range from 650 nm to 1000 nm (e.g., wherein the light consists essentially of light having a wavelength of substantially 670 nm and/or substantially 830 nm) into an eye (e.g., cornea or retina) and/or onto a skin of a subject by a display (e.g., a backlight or front light of the display) (e.g., in combination with a separate pharmaceutical composition, e.g. given as an ocular or topical administration). The condition can be inflammation, cell degeneration, visual decline (e.g., age-related vision loss), glaucoma, optic nerve injury, ocular implant recovery, increased intraocular pressure, retinitis pigmentosa, or seasonal affective disorder. Administration can occur while the subject receives information (e.g., watches a television show or film or reads text) from the display.

In some embodiments, a method for stimulating generation of adenosine triphosphate (ATP) in a subject comprises administering light (e.g., light therapy) to a subject by directing light predominately (e.g., at least 50%, at least 80%, or at least 90%) or solely having one or more wavelengths in a range from 650 nm to 1000 nm (e.g., wherein the light consists essentially of light having a wavelength of substantially 670 nm and/or substantially 830 nm) into an eye (e.g., cornea or retina) of a subject by a display (e.g., a backlight or front light of the display) such that a therapeutically effective amount of light is delivered to the subject to stimulate the generation of ATP (e.g., in combination with a separate pharmaceutical composition, e.g. given as an ocular or topical administration). In some embodiments, administration occurs while the subject receives information (e.g., watches a television show or film or reads text) from the display.

According to some embodiments of the present disclosure, therapeutic light emitted by therapeutic-light emitter 40 is visible light, for example red light and is emitted at a level that is noticeable to a viewer. Such light emission can be a part of the light emitted by a pixel light emitter 30. For example, if the information signal requires red-light emission by the pixel light emitters 30, that red light can be chosen to be therapeutic light. Thus, according to some embodiments of the present disclosure, a color display comprises pixels incorporating a red-light emitter that emits red therapeutic light. In some embodiments, light emitted by a red pixel light emitter 30 is converted to a red therapeutic light, for example with color conversion phosphors or quantum dots, for example using the structures illustrated in FIGS. 5A and 5B. Thus, according to embodiments of the present disclosure, a therapeutic information display 99 comprises an array of pixels 20 that display information. Each pixel 20 in the array of pixels 20 comprises one or more pixel light emitters 30 and one of the pixel light emitters is a therapeutic-light emitter 40. The therapeutic-light emitter 40 emits light having a wavelength between 650 and 1000 nm. In some embodiments, each pixel 20 comprises a red-light emitter (e.g., a red pixel light emitter 30R) and the red-light emitter is a therapeutic-light emitter 40, for example as shown in FIG. 3. In some embodiments, each pixel 20 comprises a therapeutic-light emitter 40 and a red-light emitter (e.g., red pixel light emitter 30R) in addition to therapeutic-light emitter 40, for example as shown in FIG. 8.

In some embodiments, a therapeutic-light emitter 40 that emits visible therapeutic red light is not controlled by the information signal as part of pixel 20. Instead, for display images that have red in the image, therapeutic-light emitter 40 emits the portion of light that is red and the pixel is controlled to emit less red. For example if an image has red at every pixel 20 location, that red can be emitted by therapeutic-light emitter 40 and the intensity of red light that is emitted by therapeutic-light emitter 40 is not emitted by pixels 20. If some areas of the display image have a greater amount of red, that greater amount of light can be emitted by red pixel light emitters 30R. In some embodiments, for example as illustrated in FIG. 4, where multiple therapeutic-light emitters 40 are spatially arranged and independently controllable, areas of the image that correspond to the locations of therapeutic-light emitters 40 can be independently managed to emit red light, just as backlights 62 employ dimming zones and corresponding red pixel light emitters 30R are controlled to emit less red light to compensate for the additional red therapeutic light.

Embodiments of the present disclosure are well suited to inorganic light-emitting diode displays. Because such displays can comprise micro-transfer printed inorganic LEDs, the displays have a very low fill factor (e.g., less than 50%, less than 20%, less than 10%, or less than 5%) that provides space on a display substrate 10 to dispose therapeutic-light emitters 40 on the display substrate. Such displays are described at length in U.S. Pat. No. 9,991,163, the content of which is incorporated by reference.

Embodiments of the present disclosure comprising passive therapeutic-light emitters 40 can be applied to windows. A therapeutic window 98 can comprise a transparent sheet of material and a therapeutic-light emitter 40 comprising one or more quantum dots or phosphors embedded in the transparent sheet of material. Therapeutic-light emitter 40 emits invisible light having a wavelength no greater than 1000 nm. Therapeutic-light emitter 40 can comprise phosphors or quantum dots that convert a portion of relatively high-frequency ambient light incident therapeutic window 98 to therapeutic light. Viewers gazing through the window will therefore be exposed to therapeutic light and the window will appear slightly darker than a comparably transparent window without light-converting phosphors or quantum dots.

Exposure to red or infrared light can reduce degeneration and loss of acuity in the eye and can also be beneficial to the skin. Thus, according to embodiments of the present disclosure, viewers of therapeutic information display 99 or using a therapeutic front light 97 can benefit from the therapeutic light emitted from therapeutic-light emitter 40. Since many viewers spend many hours each day viewing information displays, using therapeutic information display 99 can improve their health, for example their eye or skin health. Similarly, viewers gazing out therapeutic windows 98 can benefit from the therapeutic light emitted from therapeutic windows 98. Thus, using therapeutic windows 98 can improve their health, for example their eye health.

Therapeutic-light emitter 40 can be or include an inorganic light emitter such as a micro-light-emitting diode having a length or width no greater than 100 microns, for example no greater than 50 microns, no greater than 20 microns, or no greater than 15 microns. Therapeutic-light emitter 40 can be micro-transfer printed from a source wafer (e.g., an LED source wafer) to a display substrate 10 or pixel substrate.

Display substrate 10 can be any suitable substrate, for example a display substrate made of glass, plastic, sapphire, quartz, ceramic, metal, or a semiconductor substrate such as are found in the integrated circuit and flat-panel display industries. Display substrate 10 can have substantially planar opposing sides, on one side of which pixels 20 and therapeutic-light emitter 40 are disposed. Display substrate 10 can be substantially transparent to light, for example at least 50% (e.g., at least 75%, 80%, 90%, 95%, or 98% or more) transparent to visible light. Display substrate 10 can be provided with electrical conductors, contact pads, and circuitry electrically connected to pixels 20 and therapeutic-light emitter 40 to control pixels 20 and therapeutic-light emitter 40.

Pixels 20 can be any of a wide variety of structures and devices and can comprise a wide variety of materials. Pixels 20 can comprise passive or active components. Pixels 20 can be electronic, optical, or optoelectronic devices, for example. Each pixel 20 can be a structure comprising multiple devices such as multiple integrated circuits or LEDs. A single integrated circuit or multiple integrated circuits can comprise a variety of materials and functional devices. Pixel light emitters 30 can be compound semiconductor inorganic light-emitting diodes.

In some embodiments, each pixel 20 can comprise a separate and independent pixel substrate on which are disposed other devices, such as red-light-emitting red micro-LEDs 30R, green-light-emitting green micro-LEDs 30G, blue-light-emitting blue micro-LEDs 30B, and a pixel controller 22. In some embodiments, therapeutic-light emitter 40 is disposed on the pixel substrate. LEDs of the present disclosure can be unpackaged die provided on corresponding source wafers and transferred from the source wafers to display substrate 10 or a pixel substrate and can therefore comprise fractured light-emitter tethers 31, as can pixel controller 22. Each LED can be transferred, for example by micro-transfer printing, to display substrate 10 and electrically connected, for example using photolithographic processes. A process of micro-transfer printing can comprise etching a cavity under each structure on the structure source wafer, leaving the structure attached to at least one anchor portion of the source wafer with at least one tether, contacting each structure with a stamp post on a stamp body of a stamp to adhere the contacted structure to the corresponding post, removing stamp and the adhered structure, fracturing the tether(s), and then contacting the structure to a target substrate, such as display substrate 10.

Embodiments of the present disclosure can be constructed by providing a display substrate 10, forming contact pads and electrical conductors on display substrate 10, transferring micro-LEDs (e.g., any one or combination of pixel light emitters 30 and therapeutic-light emitter 40) and a pixel controller 22 to display substrate 10, and photolithographically electrically connecting the micro-LEDs, pixel controller 22, and the display substrate 10 contact pads. In some embodiments display substrate 10 comprises a thin-film circuit that can control the micro-LEDs. In some embodiments, one or more of the micro-LEDs are inorganic micro-LEDs. In some embodiments, one or more of the micro-LEDs are organic micro-LEDs. In some embodiments, therapeutic information display 99 comprises a color filter and therapeutic-light emitter 40 comprises phosphors or quantum dots and is disposed in, on, or over the color filters. In some embodiments, therapeutic information display 99 is a liquid crystal display comprising a backlight and therapeutic-light emitter 40 comprises phosphors or quantum dots and is disposed in, on, or over the backlight. In some embodiments, therapeutic-light emitter 40 comprises phosphors or quantum dots that are coated on a micro-LED, for example pixel light emitters 30 or a separately controlled micro-LED that provides pump light to therapeutic-light emitter 40.

Pixel controllers 22 can be integrated circuits comprising light-emitter control circuits formed in a semiconductor structure or substrate, for example bare-die semiconductor circuits made in monocrystalline silicon using integrated circuit and photolithographic materials and methods. The semiconductor can be, for example, silicon, CMOS, or a compound semiconductor such as GaAs. Pixel controllers 22 can be micro-sized devices, for example having at least one of a length and a width less than 1000 microns (e.g., less than 500 microns, less than 250 microns, less than 100 microns, less than 50 microns, less than 20 microns, or less than 10 microns) and, optionally, a thickness less than 100 microns (e.g., less than 50 microns, less than 20 microns, less than 10 microns, or less than 5 microns). Pixel controllers 22 can be micro-transfer printable elements that are micro-transfer printed from a native source wafer and therefore can include a broken (e.g., fractured) or separated controller tether 42R.

Similarly, pixel light emitters 30R, 30G, 30B can be integrated circuits, as can therapeutic-light emitter 40, for example micro-iLEDs, formed in a semiconductor structure or substrate, for example bare-die semiconductor circuits made in monocrystalline materials such as compound semiconductors using integrated circuit and photolithographic materials and methods. The semiconductor can be, for example, a compound semiconductor such as GaN or GaAs. Light emitters 30 can be micro-sized devices, for example having at least one of a length and a width less than 1000 microns (e.g., less than 500 microns, less than 250 microns, less than 100 microns, less than 50 microns, less than 20 microns, or less than 10 microns) and, optionally, a thickness less than 100 microns (e.g., less than 50 microns, less than 20 microns, less than 10 microns, or less than 5 microns). Such monocrystalline materials can provide faster switching speeds, greater efficiency, and reduced size compared to thin-film materials found in conventional flat-panel displays. Thus, displays having devices and structures constructed according to various embodiments of the present disclosure can have improved performance and resolution. Pixel light emitters 30R, 30G, 30B can be micro-transfer printable components 30 that are micro-transfer printed from a native source wafer and therefore may include a broken or separated light-emitter tether 31. In some embodiments, pixel light emitters 30 have a height to width aspect ratio, a height to length aspect ratio, or both a height to width aspect ratio and a height to length aspect ratio of greater than 1 (e.g., greater than 1.5 or greater than 2).

Each pixel light emitter 30R, 30G, 30B or therapeutic-light emitter 40 can be, according to various embodiments, for example, a light-emitting diode (LED), an organic light-emitting diode (OLED), a micro-LED, a laser, a diode laser, or a vertical cavity surface emitting laser and can include known light-emitting diode and/or laser materials and structures. Pixel light emitters 30 and therapeutic-light emitters 40 can comprise an inorganic solid single-crystal direct bandgap light emitter, can emit visible light, such as red, green, blue, yellow, or cyan light, infrared, violet, or ultra-violet light, and can emit either coherent or incoherent light and can include phosphors, quantum dots, or other color conversion materials. Pixel light emitters 30 and therapeutic-light emitters 40 used herein can have at least one of a width from 2 to 50 μm (e.g., 2 to 5 μm, 5 to 10 μm, 10 to 20 μm, or 20 to 50 μm), a length from 2 to 50 μm (e.g., 2 to 5 μm, 5 to 10 μm, 10 to 20 μm, or 20 to 50 μm), and a height from 2 to 50 μm (e.g., 2 to 5 μm, 5 to 10 μm, 10 to 20 μm, or 20 to 50 μm). A pixel light emitter 30 or therapeutic-light emitter 40 can include or be present in one or more associated color filters, for example as described in U.S. Pat. No. 10,008,483, or one or more associated color conversion materials or articles, for example as described in U.S. Pat. No. 10,431,719.

A display 99 can be mounted in a frame.

As is understood by those skilled in the art, the terms “over” and “under” are relative terms and can be interchanged in reference to different orientations of the layers, elements, and substrates included in the present disclosure. For example, a first layer on a second layer, in some implementations means a first layer directly on and in contact with a second layer. In other implementations a first layer on a second layer includes a first layer and a second layer with another layer therebetween.

Having described certain implementations of embodiments, it will now become apparent to one of skill in the art that other implementations incorporating the concepts of the disclosure may be used. Therefore, the disclosure should not be limited to the described embodiment, but rather should be limited only by the spirit and scope of the following claims.

Throughout the description, where apparatus and systems are described as having, including, or comprising specific components, or where processes and methods are described as having, including, or comprising specific steps, it is contemplated that, additionally, there are apparatus, and systems of the disclosed technology that consist essentially of, or consist of, the recited components, and that there are processes and methods according to the disclosed technology that consist essentially of, or consist of, the recited processing steps.

It should be understood that the order of steps or order for performing certain action is immaterial so long as the disclosed technology remains operable. Moreover, two or more steps or actions in some circumstances can be conducted simultaneously. The disclosure has been described in detail with particular reference to certain embodiments thereof, but it will be understood that variations and modifications can be effected within the spirit and scope of the disclosure.

### PARTS LIST

10 display substrate/transparent sheet
11 information display
12 row wires
14 column wires
16 control signal
18 wire/trace
20 pixel
22 pixel controller
30 pixel light emitter
30R red pixel light emitter
30G green pixel light emitter
30B blue pixel light emitter
31 light-emitter tether
32 dielectric
34 contact pad
36 electrode
40 therapeutic-light emitter
50 light
60 cover substrate
62 backlight
64 liquid crystal display
97 front light
98 therapeutic window
99 therapeutic information display

1. A therapeutic information display, comprising:
an array of pixels, each pixel in the array of pixels comprising one or more pixel light emitters responsive to an information signal to emit light; and
a therapeutic-light emitter, wherein the therapeutic-light emitter emits light having a wavelength in a range of 650 nm to 1000 nm.

2. The therapeutic information display of claim 1, wherein the therapeutic-light emitter is responsive to a control signal separate from the information signal.

3. The therapeutic information display of claim 1, wherein the therapeutic-light emitter is responsive to the information signal to emit light.

4. The therapeutic information display of claim 1, comprising a plurality of therapeutic-light emitters.

5. The therapeutic information display of claim 4, wherein the number of therapeutic-light emitters is equal to the number of pixels.

6. The therapeutic information display of claim 5, wherein the number of therapeutic-light emitters is less than the number of pixels.

7. The therapeutic information display of claim 1, comprising a display substrate and wherein the pixel light emitters are disposed on the display substrate and the therapeutic-light emitter is disposed on the display substrate.

8. The therapeutic information display of claim 1, comprising a display substrate and a cover substrate and wherein the pixel light emitters are disposed on the display substrate and the therapeutic-light emitter is disposed on the cover substrate.

9. The therapeutic information display of claim 1, wherein the information display is a liquid-crystal display comprising a backlight and the backlight comprises the therapeutic-light emitter.

10-14. (canceled)

15. The therapeutic information display of claim 1, wherein the wavelength of the therapeutic-light emitter is substantially 670 nm or substantially 830 nm.

16. The therapeutic information display of claim 1, wherein the display is operable to emit the light from the therapeutic-light emitter such that one or more of (i) the light is subliminal or invisible, (ii) the light is emitted with a subliminal intensity, and (iii) the light is emitted with a duty cycle less than one.

17. The therapeutic information display of claim 1, wherein the therapeutic-light emitter is electrically connected in series with any one or more of the one or more pixel light emitters in a pixel of the array of pixels.

18. The therapeutic information display of claim 1, wherein the therapeutic-light emitter is electrically connected in parallel with any one or more of the one or more pixel light emitters in a pixel of the array of pixels.

19. The therapeutic information display of claim 1, wherein the therapeutic-light emitter is a pixel light emitter for one pixel in the array of pixels and the one pixel comprises a separate pixel light emitter that emits red light.

20. The therapeutic information display of claim 1, wherein the therapeutic-light emitter emits red light, at least one of the one or more pixel light emitters is a red light emitter, and the display comprises a controller operable to control the therapeutic-light emitter to emit red light having an intensity no greater than a least intensity of red light emitted by any red light emitter in at least one of the pixels in the array of pixels.

21. The therapeutic information display of claim 1, comprising a controller operable to control the therapeutic-light emitter to emit light only when one or more of the pixel light emitters are controlled to emit red light wherein the therapeutic-light emitter corresponds to one or more of the one or more of the pixel light emitters.

22.-23. (canceled)

24. A therapeutic information display, comprising an array of pixels that display information, each pixel in the array of pixels comprising one or more pixel light emitters wherein one of the pixel light emitters is a therapeutic-light emitter, and wherein the therapeutic-light emitter emits light having a wavelength between 650 and 1000 nm.

25. The therapeutic information display of claim 24, wherein each pixel comprises a red-light emitter and the red-light emitter is a therapeutic-light emitter.

26. The therapeutic information display of claim 24, wherein each pixel further comprises a second red-light emitter.

27. A therapeutic front light, comprising:
a cover substrate disposed in relation to an information display; and
a therapeutic-light emitter disposed on or in the cover substrate, wherein the therapeutic-light emitter emits invisible light having a wavelength no greater than 1000 nm.

28-52. (canceled)
